(19) United States
(12) Patent Application Publication
(10) Pub. No.: US 2010/0112099 A1
(43) Pub. Date: May 6, 2010

(54) PHYTOCHEMICAL COMPOSITIONS AND METHODS FOR ACTIVATING AMP-KINASE

(75) Inventors: Matthew L. Tripp, Gig Harbor, WA (US); John G. Babish, Brooktondale, NY (US); Linda Pacioretty, Brooktondale, NY (US); Jeffrey S. Bland, Fox Island, WA (US)

Correspondence Address: MCDERMOTT WILL & EMERY LLP, 28 STATE STREET, BOSTON, MA, 02109-1775, US

(73) Assignee: METAPROTEOMICS, LLC, San Clemente, CA (US)

(21) Appl. No.: 12/612,249

(22) Filed: Nov. 4, 2009

Related U.S. Application Data
(60) Provisional application No. 61/198,387, filed on Nov. 4, 2008.

(51) Int. Cl. A61K 36/00; A61K 36/82; A61K 36/27; A61K 36/45; A61K 36/752; A61K 36/906; A61K 38/00

(52) U.S. Cl. 424/729; 424/725; 424/732; 424/736; 424/756; 514/2

(57) ABSTRACT

The present invention relates to AMPK activation utilizing phytochemicals, natural plant extracts and combinations. Disclosed are methods, compounds, and compositions comprising drugs, medical foods, and dietary supplements for the prevention and treatment of metabolic disorders, in particular obesity, weight gain, insulin resistance syndromes, diabetes, fasting hyperlipidemia and osteoarthritis. More specifically, the invention relates to pharmaceutical therapeutic methods and compositions utilizing phytochemicals, natural plant extracts and combinations to modify myocyte, hepatocyte, adipocyte, cardiac or pancreatic physiology through activation of AMPK.

### CROSS-REFERENCE TO RELATED APPLICATIONS

This patent application claims priority to U.S. provisional application Ser. No. 61/198,387, filed on Nov. 4, 2008. The contents of the priority application are incorporated herein by reference in their entirety as though fully set forth herein.

### BACKGROUND OF THE INVENTION

1. Field of the Invention

The present invention provides compositions, methods, compounds, and compositions comprising drugs, medical foods, and dietary supplements for the prevention and treatment of metabolic disorders, in particular obesity, weight gain, insulin resistance syndromes, diabetes, fasting hyperlipidemia and osteoarthritis. More specifically, the invention relates to pharmaceutical therapeutic methods and compositions utilizing such compositions to modify myocyte, hepatocyte, adipocyte, cardiac or pancreatic physiology to activate AMP-kinase (AMPK). The present invention also relates to the use of the compounds of this invention for the treatment of obesity-related diseases including associated dyslipidemia and other obesity- and overweight-related complications such as, for example, cholesterol gallstones, gallbladder disease, gout, cancer (e.g., colon, rectum, prostate, breast, ovary, endometrium, cervix, gallbladder, and bile duct), menstrual abnormalities, infertility, polycystic ovaries, osteoarthritis, and sleep apnea, as well as for a number of other pharmaceutical uses associated therewith, such as the regulation of appetite and food intake, dyslipidemia, hypertriglyceridemia, Syndrome X, type 2 diabetes (non-insulin-dependent diabetes), atherosclerotic diseases such as heart failure, hyperlipidemia, hypercholesteremia, low HDL levels, hypertension, cardiovascular disease (including atherosclerosis, coronary heart disease, coronary artery disease, and hypertension), cerebrovascular disease such as stroke, and peripheral vessel disease. The compounds of this invention may also be useful for treating physiological disorders related to, for example, regulation of insulin sensitivity, inflammatory response, plasma triglycerides, HDL, LDL and cholesterol levels and the like.

2. Description of the Related Art

Obesity is a disease resulting from a prolonged positive imbalance between energy intake and energy expenditure. In 2000, an estimated 30.5% of adults in the U.S. were obese (i.e. had a body mass index [BMI] greater than 30 kg/m²) and 15.5% of adolescents were overweight (BMI of 25 to 30 kg/m²). Excess body weight is one of the most important risk factors for all-cause morbidity and mortality. The likelihood of developing conditions such as type 2 diabetes, heart disease, cancer and osteoporosis of weight-bearing joints increases with body weight. The rapidly increasing world-wide incidence of obesity and its association with serious comorbid diseases means it is beginning to replace undernutrition and infectious diseases as the most significant contributor to ill health in the developed world.

In general terms, obesity is the result of caloric intake exceeding caloric expenditure over an extended period. Thus, obesity may be addressed by decreasing food intake, increasing energy expenditure or a combination of both. Selected modulators of food intake include: (1) Arachidonoylethanolamide (AEA; Anandamide) an endogenous cannabinoid neurotransmitter found in animal and human organs, especially in the brain; functions through G-protein coupled receptors (GPCR) termed CB1; (2) Orexin A and B peptides suggested to be primarily involved in the stimulation of food intake; (3) Neuropeptide Y—a 36 amino acid peptide neurotransmitter found in the brain and autonomic nervous system; it augments the vasoconstrictor effects of noradrenergic neurons. NPY has been associated with a number of physiologic processes in the brain, including the regulation of energy balance, memory and learning, and epilepsy; (4) Melanin-concentrating hormone (MCH)—cyclic orexinogenic hypothalamic peptide originally isolated from the pituitary gland of teleost fish where it controls skin pigmentation; in mammals it is involved in the regulation of feeding behavior and energy balance; (5) Peptide YY functions through neuropeptide Y receptors, inhibits gastric motility and increases water and electrolyte absorption in the colon; it is secreted by the gut in response to a meal, and has been shown to reduce appetite; and (6) Norepinephrine—activates the α1, α2 β1, β2 and β3 and adrenergic receptors of sympathetic nervous system to directly increase heart rate, release energy from glucose and glycogen, increase muscle readiness and induce lipolysis from adipocytes.

The 5-AMP-activated protein kinase (AMPK) functions as an intracellular fuel sensor that affects metabolism and gene expression in humans and rodents (FIG. 1). AMPK has been described as an integrator of regulatory signals monitoring systemic and cellular energy status. Recently, it has been proposed that AMPK could provide a link in metabolic defects underlying progression to the metabolic syndrome. AMPK is a heterotrimeric enzyme complex consisting of a catalytic subunit alpha and two regulatory subunits beta and gamma. AMPK is activated by rising AMP and falling ATP. AMP activates the system by binding to the gamma subunit that triggers phosphorylation of the catalytic alpha subunit by the upstream kinases LKB 1 and CaMKKbeta (calmodulin-dependent protein kinase kinase). AMPK system is a regulator of energy balance that, once activated by low energy status, switches on ATP-producing catabolic pathways (such as fatty acid oxidation and glycolysis), and switches off ATP-consuming anabolic pathways (such as lipogenesis), both by short-term effect on phosphorylation of regulatory proteins and by long-term effect on gene expression (FIG. 2).

As well as acting at the level of the individual cell, the system also regulates food intake and energy expenditure at the whole body level, in particular by mediating the effects of insulin sensitizing adipokines leptin and adiponectin. AMPK is robustly activated during skeletal muscle contraction and myocardial ischemia playing a role in glucose transport and fatty acid oxidation. In liver, activation of AMPK results in enhanced fatty acid oxidation as well as decreased glucose production [Viollet, B., Mounier, R., Leclerc, J., Yazigi, A., Foretz, M., and Andreelli, F. Targeting AMP-activated protein kinase as a novel therapeutic approach for the treatment of metabolic disorders. Diabetes Metab 2007, 33, 395-402]. The net effect of AMPK activation is stimulation of hepatic fatty acid oxidation and ketogenesis, inhibition of cholesterol synthesis, lipogenesis, and triglyceride synthesis, inhibition of adipocyte lipolysis and lipogenesis, stimulation of skeletal muscle fatty acid oxidation and muscle glucose uptake, and modulation of insulin secretion by pancreatic beta-cells (FIG. 3).

AICAR 5-Aminoimidazole-4-carboxamide ribonucleoside (AICAR) represents a useful tool for identifying new target pathways and processes regulated by the AMPK protein kinase cascade. Incubation of rat hepatocytes with AICAR results in accumulation of the monophosphorylated derivative (5-aminoimidaz-ole-4-carboxamide ribonucleoside; ZMP) within the cell. ZMP mimics both activating effects of AMP on AMPK, i.e. direct allosteric activation and promotion of phosphorylation by AMPK. kinase. Unlike existing methods for activating AMPK in intact cells (e.g. fructose, heat shock), AICAR does not perturb the cellular contents of ATP, ADP or AMP. Incubation of hepatocytes with AICAR activates AMPK due to increased phosphorylation, causes phosphorylation and inactivation of a known target for AMPK (3-hydroxy-3-methylglutaryl-CoA reductase), and almost total cessation of two of the known target pathways, i.e. fatty acid and sterol synthesis. Incubation of isolated adipocytes with AICAR antagonizes isoprenaline-induced lipolysis. This provides direct evidence that the inhibition by AMPK of activation of hormone-sensitive lipase by cyclic-AMP-dependent protein kinase, previously demonstrated in cell-free assays, also operates in intact cells.

Therefore additional approaches to affect sustained weight loss in obese subjects represent a critical need. Further, compounds or formulations that safely and effectively activate AMPK may function to stimulate hepatic fatty acid oxidation and ketogenesis, inhibit cholesterol synthesis, lipogenesis, and triglyceride synthesis, inhibit adipocyte lipolysis and lipogenesis, stimulate of skeletal muscle fatty acid oxidation and muscle glucose uptake, and modulate insulin secretion by pancreatic beta-cells.

### SUMMARY OF THE INVENTION

The present invention relates to the unexpected discovery that certain phytochemicals or botanical extracts activate AMPK implying stimulation of hepatic fatty acid oxidation and ketogenesis, inhibition of cholesterol synthesis, lipogenesis, and triglyceride synthesis, inhibition of adipocyte lipolysis and lipogenesis, stimulation of skeletal muscle fatty acid oxidation and muscle glucose uptake, and modulation of insulin secretion by pancreatic beta-cells.

AMPK. also regulates food intake and energy expenditure at the whole body level, in particular by mediating the effects of insulin sensitizing adipokines leptin and adiponectin. AMPK is robustly activated during skeletal muscle contraction and myocardial ischemia playing a role in glucose transport and fatty acid oxidation.

The invention provides methods for modifying myocyte, hepatocyte, adipocyte, cardiac or pancreatic physiology in a subject, comprising administering to the subject a pharmaceutical composition of phytochemical, or pharmaceutically acceptable salts or mixtures thereof. Preferred embodiments provide compositions and methods for enhancing AMPK activation utilizing either single botanical compounds or mixtures thereof.

Such modification of myocyte, hepatocyte, adipocyte, cardiac or pancreatic physiology by phytochemicals would be useful to assist in weight loss, increasing muscle mass or increasing physical performance. More particularly, the present invention relates to the unexpected discovery that the AMPK-activation potential of the phytochemicals or botanical extracts (Table 2) was similar to the well-known exercise mimetic AICAR.

One embodiment of the invention discloses methods for the treatment of obesity related disorders in a subject in need. These methods comprise administering to the subject a composition comprising a therapeutically effective amount of a pharmaceutically acceptable phytochemical formulation.

The present invention also relates to the use of the compounds of this invention for the treatment of obesity-related diseases including associated dyslipidemia and other obesity- and overweight-related complications such as, for example, cholesterol gallstones, gallbladder disease, gout, cancer (e.g., colon, rectum, prostate, breast, ovary, endometrium, cervix, gallbladder, and bile duct), menstrual abnormalities, infertility, polycystic ovaries, osteoarthritis, and sleep apnea, as well as for a number of other pharmaceutical uses associated therewith, such as the regulation of appetite and food intake, dyslipidemia, hypertriglyceridemia, Syndrome X, type 2 diabetes (non-insulin-dependent diabetes), atherosclerotic diseases such as heart failure, hyperlipidemia, hypercholesteremia, low HDL levels, hypertension, cardiovascular disease (including atherosclerosis, coronary heart disease, coronary artery disease, and hypertension), cerebrovascular disease such as stroke, and peripheral vessel disease. The compounds of this invention may also be useful for treating physiological disorders related to, for example, regulation of insulin sensitivity, inflammatory response, plasma triglycerides, HDL, LDL and cholesterol levels and the like.

### BRIEF DESCRIPTION OF THE DRAWINGS

FIG. 1 illustrates the role of ANIPK in regulating energy balance at the single-cell level. The way in which the AMPK system controls the balance between ATP consumption (e.g., by biosynthesis, cell growth, or muscle contraction) and ATP production via catabolism is illustrated. If the rate of ATP consumption exceeds its rate of production, ADP will tend to rise and be converted to AMP by the enzyme adenylate kinase. The rise in level of the activating ligand AMP, coupled with the fall in level of the inhibitory nucleotide ATP, activates AMPK, which then switches off ATP-consuming processes and switches on catabolism in an attempt to redress the balance.

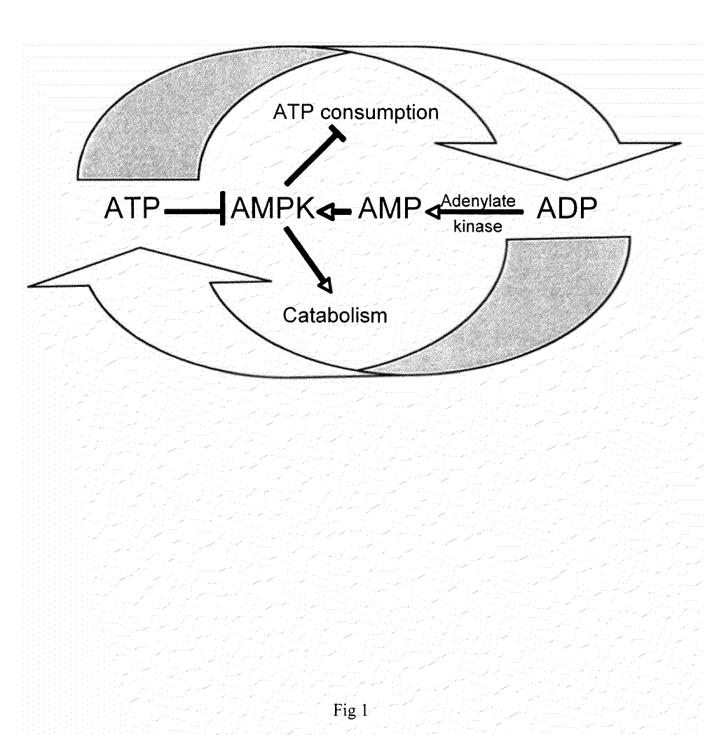

FIG. 2 is a schematic of the wide array of target proteins phosphorylated by activated AMPK.

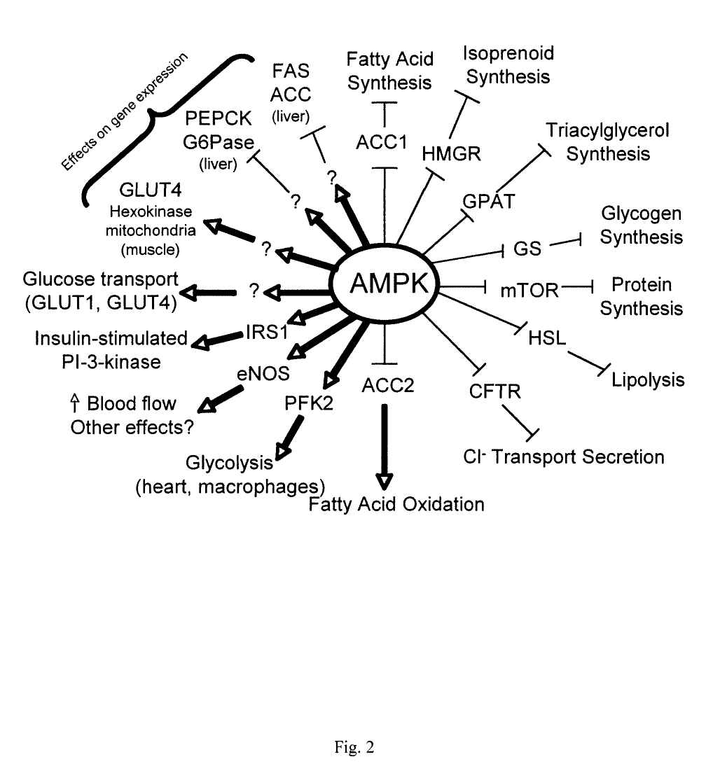

FIG. 3 depicts the role of AMPK in regulating energy balance at the whole-body level. Arrows indicate positive effects, and bars indicate negative effects. FA=fatty acid.

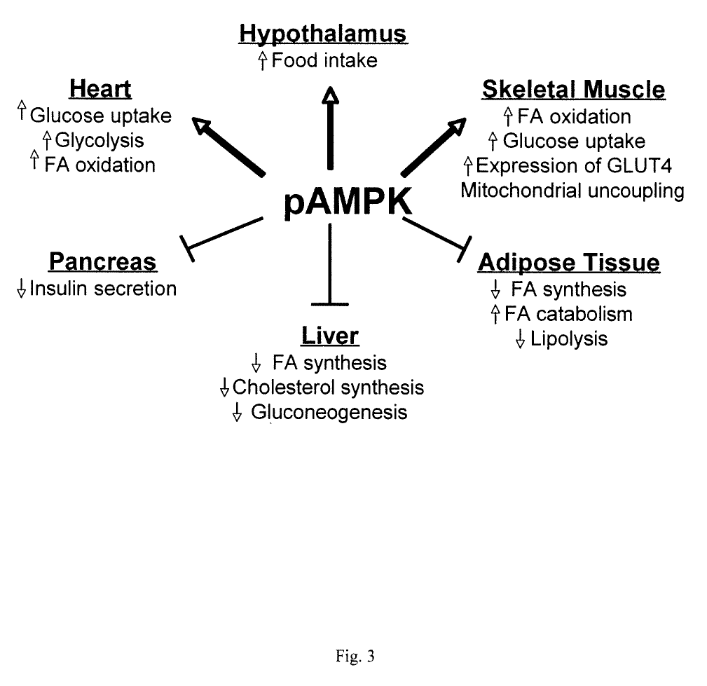

FIG. 4 illustrates the relative activation of AMPK by AICAR and three select ginger formulations in C2C12 myocytes.

### DETAILED DESCRIPTION OF THE INVENTION

The invention provides compounds, compositions, and methods for the treatment of obesity related disorders in a subject. The compositions, compounds, and methods comprise administering to the subject a composition consisting of phytochemicals or botanical extracts. The present invention relates to the unexpected discovery that the compositions described herein activate AMPK thereby increasing ATP production via catabolism resulting in increased resting energy expenditure. Preferred embodiments provide compositions, and methods for activating AMPK.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 10th Ed., McGraw Hill Companies Inc., New York (2001). Standard medical terminology used herein has the meaning defined in Stedman's Medical Dictionary, 27th Edition, with veterinary medicine insert.

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. Additionally, as used herein, unless specifically indicated otherwise, the word “or” is used in the “inclusive” sense of “and/or” and not the “exclusive” sense of “either/or.” The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2 can be 0, 1 or 2 for variables that are inherently discrete, and can be 0.0, 0.1, 0.01, 0.001, or any other real value for variables that are inherently continuous.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or compounds, but may also include additional features or compounds.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

As used herein, “myocyte, hepatocyte, adipocyte, cardiac or pancreatic modification” means a change in the physical or physiochemical function of the cell from the cell's state prior to treatment. Nonlimiting examples of physical or physiochemical functional changes include altered rates of secretion or amounts of naturally occurring secreted products, the introduction, production and secretion of novel products, the abrogation of secretion of selected compounds, or physical changes in cell morphology and function which may include alterations in membrane permeability or thickness, modification of cell surface receptor numbers or binding efficiency, or the introduction and expression of novel cell surface receptors. The methods of the invention provide for modification of myocyte physiology in a subject. While modification of myocyte, hepatocyte, adipocyte, cardiac or pancreatic physiology to enhance fatty acid oxidation is desirable in and of itself, it is to be recognized that a modification of myocyte, hepatocyte, adipocyte, cardiac or pancreatic physiology can have other salutary effects. The present compositions also reduce the inflammatory response and thereby promote healing of, or prevent further damage to, the affected tissue.

The term “treat” and its verbal variants refer to palliation or amelioration of an undesirable physiological state. Thus, for example, where the physiological state is poor glucose tolerance, “treatment” refers to improving the glucose tolerance of a treated subject. As another example, where the physiological state is obesity, the term “treatment” refers to reducing the body fat mass, improving the body mass or improving the body fat ratio of a subject. Treatment of diabetes means improvement of blood glucose control. Treatment of inflammatory diseases means reducing the inflammatory response either systemically or locally within the body. Treatment of osteoporosis means an increase in the density of bone mineralization or a favorable change in metabolic or systemic markers of bone mineralization. The person skilled in the art will recognize that treatment may, but need not always, include remission or cure.

Obesity, which is an excess of body fat relative to lean body mass, is a chronic disease that is highly prevalent in modern society. It is associated not only with a social stigma, but also with decreased life span and numerous medical problems, including adverse psychological development, coronary artery disease, hypertension, stroke, diabetes, hyperlipidemia, and some cancers. (see, e.g., Nishina, et al., Metab. 43:554-558, 1994; Grundy and Barnett, Dis. Mon. 36:641-731, 1990; Rissanen, et al., British Medical Journal, 301:835-837, 1990).

“Obesity related disorders” refers to those diseases or conditions where excessive body weight or high “body mass index (BMI)” has been implicated in the progression or suppression of the disease or condition. Representative examples of obesity related disorders include, without limitation diabetes, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia. See, Harrison's Principles of Internal Medicine, 13th Ed., McGraw Hill Companies Inc., New York (1994). Examples, without limitation, of inflammatory conditions include diseases of the digestive organs (such as ulcerative colitis, Crohn's disease, pancreatitis, gastritis, benign tumor of the digestive organs, digestive polyps, hereditary polyposis syndrome, colon cancer, rectal cancer, stomach cancer and ulcerous diseases of the digestive organs), stenocardia, myocardial infarction, sequelae of stenocardia or myocardial infarction, senile dementia, cerebrovascular dementia, immunological diseases and cancer in general.

As used herein, “AMPK-related diseases” includes pathologic or pathognomic conditions in which the activation of AMPK provides a salutary effect. Examples of such diseases or conditions include obesity, diabetes, metabolic syndrome, acute inflammatory lung injury, heart disease, reperfusion ischemia, cancer, aging, retinal degeneration, cardiac hypertrophy, non-alcoholic fatty liver disease, hypertension, albuminuria, sporadic Alzheimer's disease, muscular dystrophy, and osteoarthritis.

The term “prevent” and its variants refer to prophylaxis against a particular undesirable physiological condition. The prophylaxis may be partial or complete. Partial prophylaxis may result in the delayed onset of a physiological condition. The person skilled in the art will recognize the desirability of delaying onset of a physiological condition, and will know to administer the compositions of the invention to subjects who are at risk for certain physiological conditions in order to delay the onset of those conditions. For example, the person skilled in the art will recognize that obese subjects are at elevated risk for coronary artery disease. Thus, the person skilled in the art will administer compositions of the invention in order to increase insulin sensitivity in an obese, whereby the onset of diabetes mellitus or dyslipernia may be prevented entirely or delayed.

As used herein “obesity complications” include, without limitation, retinopathy, muscle infarction, idiopathic skeletal hyperostosis and bone loss, foot ulcers, neuropathy, arteriosclerosis, respiratory autonomic neuropathy and structural derangement of the thorax and lung parenchyma, left ventricular hypertrophy, cardiovascular morbidity, progressive loss of kidney function, and anemia.

As used herein, the term “fasting hyperlipidemia” refers to a pathognomonic condition manifest by elevated serum concentrations of total cholesterol (>200 mg/dL), LDL cholesterol (>130 mg/dL), or triglycerides (>150 mg/dL) or decreased HDL cholesterol (<40 mg/dL). Further, as used herein, the term ‘fat” refers to serum and adipose triglyceride content and “triglycerides” refers to triacylglycerol esters of fatty acids.

As used herein, the terms hyperinsulinemia” and “hyperglycemia” refer to a fasting insulin concentration >17 IU/ml) and fasting glucose >125 mg/dL.

As used herein, the term “impaired fasting glucose” refers to fasting serum glucose values greater than 110 mg/dL measured on at least two separate occasions.

As used herein, the term “insulin sensitivity” refers to the ability of a cell, tissue, organ or whole body to absorb glucose in response to insulin. As used in an in vivo context, “insulin sensitivity” refers to the ability of an organism to absorb glucose from the blood stream. An improvement in insulin sensitivity therefore results in an improved ability of the organism to maintain blood glucose levels within a target range. Thus, improved insulin sensitivity may also result in a decreased incidence of hyperglycemia. Improved insulin sensitivity can also treat, prevent or delay the onset of various metabolic conditions, such as diabetes mellitus, syndrome X and diabetic complications. Because of the improved metabolic processing of dietary sugar, improved insulin sensitivity can also treat, prevent or delay the onset of hyperlipidemia and obesity. Additionally, improved insulin sensitivity can lead to treatment, prevention or delayed onset of a variety of inflammatory conditions, such as, for example, diseases of the digestive organs (such as ulcerative colitis, Crohn's disease, pancreatitis, gastritis, benign tumor of the digestive organs, digestive polyps, hereditary polyposis syndrome, colon cancer, rectal cancer, stomach cancer and ulcerous diseases of the digestive organs), stenocardia, myocardial infarction, sequelae of stenocardia or myocardial infarction, senile dementia, cerebrovascular dementia, immunological diseases and cancer in general.

In regard to improvement of insulin sensitivity, then, a subject may be an animal or human who has been diagnosed with insulin resistance or an animal or human, such as an ordinary clinician will be able to diagnose insulin resistance and, via analysis of a subject's health history, determine whether the subject is at risk for insulin resistance.

The methods of the present invention are intended for use with any subject that may experience the benefits of the methods of the invention. Thus, in accordance with the invention, “subjects” include humans as well as non-human subject, particularly domesticated animals. It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses.

As used herein, “improved secretion,” means to increase by at least 3%, the rate of secretion or amount of secretion of the referent compound. The invention further provides a method of improving plasma adiponectin concentrations in a subject, comprising administering to the subject an amount of the compound or composition sufficient to increase adiponectin secretion from adipocytes in the subject.

In general, an increase in tissue AMPK activation will result in improved insulin sensitivity resulting in improved glucose metabolism, improved blood lipid profiles, and decreased pro-inflammatory cytokine secretion. A decrease in pro-inflammatory cytokine secretion leads to decreased systemic inflammation and disorders associated with inflammation, such as diabetic complications, obesity, inflammatory diseases of the digestive organs, proliferative diseases of the digestive organs, ulcerous diseases of the digestive organs, stenocardia, myocardial infarction, sequelae of stenocardia, sequelae of myocardial infarction, senile dementia, cerebrovascular dementia, immunological diseases and cancer.

In some aspects the compositions further comprise a pharmaceutically acceptable excipient where the pharmaceutically acceptable excipient is selected from the group consisting of coatings, isotonic and absorption delaying agents, binders, adhesives, lubricants, disintegrants, coloring agents, flavoring agents, sweetening agents, absorbents, detergents, and emulsifying agents. In yet further aspects, the composition additionally comprises one or more members selected from the group consisting of antioxidants, vitamins, minerals, proteins, fats, and carbohydrates.

The term “therapeutically effective amount” is used to denote treatments at dosages effective to achieve the therapeutic result sought. Furthermore, one of skill will appreciate that the therapeutically effective amount of the compound of the invention may be lowered or increased by fine-tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined, for example, empirically by starting at relatively low amounts and by step-wise increments with concurrent evaluation of beneficial effect.

The term “pharmaceutically acceptable” is used in the sense of being compatible with the other ingredients of the compositions and not deleterious to the recipient thereof.

As used herein, “compounds” may be identified either by their chemical structure, chemical name, or common name. When the chemical structure and chemical or common name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated or identified compounds. The compounds described also encompass isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. Also contemplated within the scope of the invention are congeners, analogs, hydrolysis products, metabolites and precursor or prodrugs of the compound. In general, all physical forms are equivalent for the uses contemplated herein and are intended to be within the scope of the present invention.

The compounds according to the invention are optionally formulated in a pharmaceutically acceptable vehicle with any of the well-known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well any other pharmacologically active ingredient useful for the treatment of the symptom/condition being treated.

The compounds of the present invention may be provided in a pharmaceutically acceptable vehicle using formulation methods known to those of ordinary skill in the art. The compositions of the invention can be administered by standard routes. The compositions of the invention include those suitable for oral, inhalation, rectal, ophthalmic (including intravitreal or intracameral), nasal, topical (including buccal and sublingual), vaginal, or parenteral (including subcutaneous, intramuscular, intravenous, intradermal, and intratracheal). In addition, polymers may be added according to standard methodologies in the art for sustained release of a given compound.

It is contemplated within the scope of the invention that compositions used to treat a disease or condition will use a pharmaceutical grade compound and that the composition will further comprise a pharmaceutically acceptable carrier. It is further contemplated that these compositions of the invention may be prepared in unit dosage forms appropriate to both the route of administration and the disease and patient to be treated. The compositions may conveniently be presented in dosage unit form be prepared by any of the methods well known in the art of pharmacy. All methods include the step of bringing the active ingredient into association with the vehicle that constitutes one or more auxiliary constituents. In general, the compositions are prepared by uniformly and intimately bringing the active ingredient into association with a liquid vehicle or a finely divided solid vehicle or both, and then, if necessary, shaping the product into the desired composition.

The term “dosage unit” is understood to mean a unitary, i.e. a single dose which is capable of being administered to a patient, and which may be readily handled and packed, remaining as a physically and chemically stable unit dose comprising either the active ingredient as such or a mixture of it with solid or liquid pharmaceutical vehicle materials.

Compositions suitable for oral administration may be in the form of discrete units as capsules, sachets, tablets, soft gels or lozenges, each containing a predetermined amount of the active ingredient; in the form of a powder or granules; in the form of a solution or a suspension in an aqueous liquid or non-aqueous liquid, such as ethanol or glycerol; or in the form of an oil-in-water emulsion or a water-in-oil emulsion. Such oils may be edible oils, such as e.g. cottonseed oil, sesame oil, coconut oil or peanut oil. Suitable dispersing or suspending agents for aqueous suspensions include synthetic or natural gums such as tragacanth, alginate, gum arabic, dextran, sodium carboxymethylcellulose, gelatin, methylcellulose and polyvinylpyrrolidone. The active ingredient may also be administered in the form of a bolus, electuary or paste.

Transdermal compositions may be in the form of a plaster, microstructured airways, sometimes called microneedles, iontophoresis (which uses low voltage electrical current to drive charged drugs through the skin), electroporation (which uses short electrical pulses of high voltage to create transient aqueous pores in the skin), sonophoresis (which uses low frequency ultrasonic energy to disrupt the stratum corneum), and thermal energy (which uses heat to make the skin more permeable and to increase the energy of drug molecules), or via polymer patch.

Compositions suitable for ophthalmic administration may be in the form of a sterile aqueous preparation of the active ingredients, which may be in microcrystalline form, for example, in the form of an aqueous microcrystalline suspension. Liposomal compositions or biodegradable polymer systems may also be used to present the active ingredient for ophthalmic administration.

Compositions suitable for topical or ophthalmic administration include liquid or semi-liquid preparations such as liniments, lotions, gels, and oil-in-water or water-in-oil emulsions such as creams, ointments or pastes; or solutions or suspensions such as drops.

In addition to the compositions described above, the compositions of the invention may also be formulated as a depot preparation. Such long-acting compositions may be administered by implantation (e.g. subcutaneously, intraabdominally, or intramuscularly) or by intramuscular injection. Thus, for example, the active ingredient may be formulated with suitable polymeric or hydrophobic materials (for example, as an emulsion in a pharmaceutically acceptable oil), or an ion exchange resin.

For systemic treatment according to the present invention, daily doses of phytochemicals or botanical extracts from 0.001-200 mg/kg body weight, preferably from 0.002-20 mg/kg of body weight, for example 0.003-10 mg/kg of the combination are administered, corresponding to a daily dose for an adult human of from 0.2 to 14000 mg of the active ingredients or marker compounds. In the topical treatment of dermatological disorders, ointments, creams or lotions containing from 0.1-750 mg/g, and preferably from 0.1-500 mg/g, of the combination may be administered. For topical use in opthalmological ointments, drops or gels containing from 0.1-750 mg/g, and preferably from 0.1-500 mg/g, of the formulation are administered. Oral compositions are formulated, preferably as tablets, capsules, or drops, containing from 0.05-250 mg, preferably from 0.1-1000 mg, of the formulation per dosage unit.

The compounds of this invention either alone or in combination with each other or other compounds generally will be administered in a convenient composition. The following representative composition examples are illustrative only and are not intended to limit the scope of the present invention. In the compositions that follow, “active ingredient” means a compound of this invention.

As used herein, “regulating insulin levels or sensitivity” refers to means for maintaining insulin levels at a particular value or inducing a desired change (either increasing or decreasing) in the level of insulin or in the response to endogenous or exogenous insulin.

As used herein, “therapeutically effective time window” means the time interval wherein administration of the compounds of the invention to the subject in need thereof reduces or eliminates the deleterious effects or symptoms. In a preferred embodiment, the compound of the invention is administered proximate to the deleterious effects or symptoms.

The term “extract” refers to the solid material resulting from (1) exposing a botanical to a solvent, (2) separating the solvent from the plant products, and (3) removing the solvent.

Preferably, a daily dose of the present composition would be formulated to deliver about 0.05 to 20 g of phytochemical or botanical extract per day.

More preferably, an effective daily dose of the present composition would be formulated to deliver about 0.01 to 15,000 mg of phytochemical or botanical extract per day.

Further Ingredients—The formulation can also contain other ingredients such as one or a combination of other vitamins, minerals, antioxidants, fiber and, other nutritional supplements. Selection of one or several of these ingredients is a matter of formulation design, consumer and end-user preference. The amount of these ingredients added to the nutritional supplements of this invention are readily known to the skilled artisan and guidance to such amounts can be provided by the RDA (Recommended Dietary Allowance) and DRI (Dietary Reference Intake) doses for children and adults. Vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacin amide; zinc sulfate or oxide; calcium pantothenate; copper gluconate; riboflavin; beta-carotene; pyridoxine hydrochloride; thiamin mononitrate; folic acid; biotin; potassium iodide; selenium; sodium selenate; sodium molybdate; phylloquinone; Vitamin D₃; cyanocobalamin; sodium selenite; copper sulfate; Vitamin A; Vitamin E; vitamin B₆ and hydrochloride thereof; Vitamin C; inositol; Vitamin B₁₂ and potassium iodide.

The amount of other additives per unit serving are a matter of design and will depend upon the total number of unit servings of the nutritional supplement daily administered to the patient. The total amount of other ingredients will also depend, in part, upon the condition of the patient. Preferably, the amount of other ingredients will be a fraction or multiplier of the RDA or DRI amounts. For example, the nutritional supplement will comprise 50% RDI (Reference Daily Intake) of vitamins and minerals per unit dosage and the patient will consume two units per day.

Flavors, coloring agents, spices, nuts and the like can be incorporated into the product. Flavorings can be in the form of flavored extracts, volatile oils, chocolate flavorings (e.g., non-caffeinated cocoa or chocolate, chocolate substitutes such as carob), peanut butter flavoring, cookie crumbs, crisp rice, vanilla or any commercially available flavoring. Flavorings can be protected with mixed tocopherols. Examples of useful flavorings include but are not limited to pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, cherry oil, walnut oil, cinnamon oil, clove oil, or peppermint oil; peanut butter, chocolate flavoring, vanilla cookie crumb, butterscotch or toffee. In a preferred embodiment, the nutritional supplement contains berry or other fruit flavor. The food compositions may further be coated, for example with a yogurt coating if it is as a bar.

Emulsifiers may be added for stability of the final product. Examples of suitable emulsifiers include, but are not limited to, lecithin (e.g., from egg or soy), or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutritional supplement can contain natural or artificial sweeteners, e.g., glucose, sucrose, fructose, saccharides, cyclamates, aspartamine, sucralose, aspartame, acesulfame K, or sorbitol.

Manufacture of the Preferred Embodiments—The nutritional supplements of the present invention may be formulated using any pharmaceutically acceptable forms of the vitamins, minerals and other nutrients discussed above, including their salts. They may be formulated into capsules, tablets, powders, suspensions, gels or liquids optionally comprising a physiologically acceptable carrier, such as but not limited to water, milk, juice, soda, starch, vegetable oils, salt solutions, hydroxymethyl cellulose, carbohydrate. In a preferred embodiment, the nutritional supplements may be formulated as powders, for example, for mixing with consumable liquids, such as milk, juice, sodas, water or consumable gels or syrups for mixing into other nutritional liquids or foods. The nutritional supplements of this invention may be formulated with other foods or liquids to provide pre-measured supplemental foods, such as single serving beverages or bars, for example.

In a particularly preferred embodiment, the nutritional supplement will be formulated into a nutritional beverage, a form that has consumer appeal, is easy to administer and incorporate into one's daily regimen, thus increasing the chances of patient compliance. To manufacture the beverage, the ingredients are dried and made readily soluble in water. For the manufacture of other foods or beverages, the ingredients comprising the nutritional supplement of this invention can be added to traditional formulations or they can be used to replace traditional ingredients. Those skilled in food formulating will be able to design appropriate foods or beverages with the objective of this invention in mind.

The nutritional supplement can be made in a variety of forms, such as puddings, confections, (i.e., candy), nutritional beverages, ice cream, frozen confections and novelties, or non-baked, extruded food products such as bars. The preferred form is a powder to add to a beverage or a non-baked extruded nutritional bar. In another embodiment, the ingredients can be separately assembled. For example, certain of the ingredients (e.g., black current PE 10%, Acacia nilotica, or 6-gingerol) can be assembled into a tablet or capsule using known techniques for their manufacture. The remaining ingredients can be assembled into a powder or nutritional bar. For the manufacture of a food bar, the dry ingredients are added with the liquid ingredients in a mixer and mixed until the dough phase is reached; the dough is put into an extruder and extruded; the extruded dough is cut into appropriate lengths; and the product is cooled. The two assembled forms comprise the nutritional supplement and can be packaged together or separately, such as in the form of a kit, as described below. Further, they can be administered together or separately, as desired.

Use of Preferred Embodiments—The preferred embodiments contemplate treatment of obesity related disorder selected from the group consisting of body weight gain, diabetes, diabetic complications, insulin sensitivity, hyperglycemia, dyslipidemia, insulin resistance, metabolic syndrome. A pharmaceutically acceptable carrier may also be used in the present compositions and formulations.

The preferred embodiments are directed to the treatment of human beings the to treat an obesity related disorder selected from the group consisting of diabetes, diabetic complications, insulin sensitivity, hyperglycemia, dyslipidemia, insulin resistance, metabolic syndrome, and body weight gain. Administration can be by any method available to the skilled artisan, for example, by oral, transmucosal, or parenteral routes. The composition and nutritional supplements of the invention are intended to be orally administered daily. Based on the serving size of 1.5-2.0 g powder in 8 oz. water, the recommended dosage is once daily. For example, if the supplement is in the form of a beverage or food bar, then the patient would consume the composition before, after or during the largest meal. The recommended daily amounts of each ingredient, as described above, serve as a guideline for formulating the nutritional supplements of this invention. The actual amount of each ingredient per unit dosage will depend upon the number of units daily administered to the individual in need thereof. This is a matter of product design and is well within the skill of the nutritional supplement formulator.

The ingredients can be administered in a single formulation or they can be separately administered. For example, it may be desirable to administer the compounds in a form that masks their taste (e.g., capsule or pill form) rather than incorporating them into the nutritional composition itself (e.g., powder or bar). Thus, the invention also provides a pharmaceutical pack or kit comprising one or more containers filled with one or more of the ingredients of the nutritional compositions of the invention (e.g., nutritional supplement in the form of a powder and capsules containing phytochemical or botanical extract). Optionally associated with such container(s) can be a notice in the form prescribed by a government agency regulating the manufacture, use or sale of pharmaceutical products, which notice reflects approval by the agency of manufacture, use of sale for human administration. The pack or kit can be labeled with information regarding mode of administration, sequence of administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a formulation or tablet.

The preferred embodiments provide compositions and methods to promote fat redistribution, resting energy expenditure or decrease fasting hyperlipidemia in any subject in need thereof.

In some aspects of this embodiment of the invention, the compositions are useful for modification of myocyte, hepatocyte, adipocyte, cardiac or pancreatic physiology to enhance fatty acid oxidation. It is, however, to be recognized that a modification of myocyte, hepatocyte, adipocyte, cardiac or pancreatic physiology can have other salutary effects. The present compositions also reduce the inflammatory response and thereby promote healing of, or prevent further damage to, affected tissues as previously disclosed.

### EXAMPLES

### Example 1

### AICAR Activates AMPK in C2C12 Myocytes

Objective—The objective of this experiment was to observe the effect of the AMP mimetic AICAR on AMPK activation in C2C12 myocytes.

The Model—The C2C12 myocyte model is commonly used to study the potential effects of compounds on muscle tissue in vitro.

Chemicals—Penicillin, streptomycin, Dulbecco's modified Eagle's medium (DMEM) was from Mediatech (Herndon, Va.) and 10% FBS-HI (fetal bovine serum-heat inactivated) from Mediatech and Hyclone (Logan, Utah). Unless noted, all other standard reagents were purchased from Sigma (St. Louis, Mo.).

Cell culture—Mouse C2C12 myoblasts were obtained from American Type Culture Collection (Manassas, Va.), and were maintained in Dulbecco's modified Eagle's medium (DMEM) supplemented with 10% fetal bovine serum at 37° C. under a humidified atmosphere of 5% CO₂.

C2C12 cells were seeded at an initial density of 6×10⁴ cells/cm² in 24-well plates. For two days, the cells were allowed grow to reach confluence. Following confluence, the cells were forced to differentiate into myocytes by culturing in DMEM supplemented with 2% horse serum for seven days.

Treatment with AICAR—On Day 8 to 10 post differentiation, C2C12 myocytes were incubated in serum-free DMEM plus 0.5% BSA (bovine serum albumin) for three hours. Next, AICAR (Cell Signal, Danvers, Mass.) was dissolved in phosphate buffered saline (PBS) and added to the culture medium to achieve concentrations of 1 mM per column (4 replicates) for 30 min at 37° C.

Measuring activated AMPKα—pT¹⁷²-AMPK was quantified using the Biosource AMPKa Immunoassay Kit (Camarillo, Calif.) without modification. Protein content of the cell lysates was determined with the Active Motif fluorescent protein assay reagent (Carlsbad Calif., Hoefelschweiger, B. K., Duerkop, A., and Wolfbeis, O, S, Novel type of general protein assay using a chromogenic and fluorogenic amine-reactive probe. Anal Biochem 2005, 344, 122-9). A Packard Fluorocount spectrofluorometer (Model#BF10000, Meridan, Conn.) was used for protein determination and a MEL312e BIO-KINETICS READER (Bio-Tek Instruments, Winooski, Vt.) was used for quantification of pT¹⁷²-AMPK.

Calculation of relative activation of AMPK —pT¹⁷²-AMPK was computed per mg lysate protein and then normalized to the dimethyl sulfoxide (DMSO) negative controls. For statistical comparisons, 95% confidence intervals were computed (Excel, Microsoft, Redman, Wash.).

Results—Over ten independent assays, 1 mM AICAR increased pT¹⁷²-AMPK an average of 1.67-fold (95% CI=1.26-2.21) in C2C12 myocytes relative to the DMSO negative controls.

### Example 2

### Select Phytochemicals and Botanical Extracts Activate AMPK in C2C12 Myocytes

Objective—The objective of this experiment was to determine the ability of phytochemicals or botanical extracts to activate AMPK in C2C12 myocytes relative to AICAR activation.

The Model—The C2C12 murine myocyte model as described in Example 1 was used.

Chemicals, Cell Culture and Treatment—Chemicals, cell culture procedures, methods and statistical procedures used were as noted in Example 1.

Test Materials—Phytochemicals or botanical extracts as described in Table 1 were used as the test materials and dosed at 25 μglmL. The concentration for the positive control AICAR run concurrently was 1.0 mM (338 μg/mL).

TABLE 1
Commercial Sources of Test Materials
Used in AMPK Activation Assays

| Test Material | Commercial Source |
|---|---|
| 6-Gingerol | Sigma, St. Louis, MO |
| Acacia nilotica | Indfrag-KDN Vita, Hillsborough, |
| | NJ |
| Acai 10:1 | DNP, Whittier, CA |
| Advantra Z 30% | Nutratech, Pompton Plains, NJ |
| Anethole | Sigma, St. Louis, MO |
| Applephenon | A. M. Todd, Logan, UT |
| AppleZin | Cyvex Nutrition, Irvine, CA |
| Bayberry Bark PE (80% Flavones) | Cactus Botanics, Long Beach, CA |
| BCM-95 | Dolcas Biotech, Chester, NJ |
| Berberine | Sigma, St. Louis, MO |
| Black Currant PE 10% | DNP, Whittier, CA |
| Black rice extract 15% | Draco, San Jose, CA |
| Black Tea Extract | CBC, Commack, NY |
| Black Tea PE 60% | Naturex, South Hakensack, NJ |
| Blueberry Leaf PE 20% | Naturex, South Hakensack, NJ |
| Chlorogenic acid | |
| Cascara Sagrada Bark Powder | American Ingredients, Anaheim, CA |
| Cassia Nomame extract powder | Novel Ingred, West Caldwell, NJ |
| Cha de Bugre PE 10:1 | NP Nutra, Gardena, CA |
| Chromium polynicotinate | American Ingredients, Anaheim, CA |
| (0.5% Cr) | |
| Cissus quadrangularis | Verdure Sciences/Geni Herbs, |
| | Noblesville, IN |
| Cocoanox 45% Polyphenols | P L Thomas, Morristown, NJ |
| Cranberry 90 MX Pwd | Ocean Spray, Lakeville/Middleboro, |
| | MA |
| Elderberry Dry Ext. 4:1 | Suan Farma, Hakensack, NJ |
| Emblica officinalis (Amla) | Verdure Sciences/Geni Herbs, |
| | Noblesville, IN |
| Epimediurn (Horny Goat Weed) | Naturex, South Hakensack, NJ |
| PE 20% | |
| Exxenterol | Suan Farma, Hakensack, NJ |
| Fenugreek Extract Pwd | Gencor Pacific, Anaheim, CA |
| Fisetin - Cotinus coggygria | Novel Ingred, West Caldwell, NJ |
| Ext Pwd 10% | |
| Green Coffee Bean Ext | AFS, Austin, TX |
| Green Coffee Dry Ext. | Suan Farma, Hakensack, NJ |
| Green Tea 98% Polyphenols/ | Maypro, Purchase, NY |
| 80% EGCG | |
| Green Tea Extract | Maypro, Purchase, NY |
| GSE (grape seed extract, | Polyphenolics (P L Thomas), |
| MegaNatural) | Morriston, NJ |
| Gymnema sylvesre ext | Kancor, Short Hills, NJ |
| Hexahydroisoalpha acids | Metagenics, Gig Harbor, WA |
| (HHIAA) | |
| ID-alG | Bio serae (Charles Bowman), |
| | Holland, MI |
| Insinase | Metagenics, Gig Harbor, WA |
| Isoalpha acids (IAA) | Metagenics, Gig Harbor, WA |
| Jambolean 10% | Roxlor International, Wilmington, |
| | DE |
| Licorice Root 26% | P L Thomas, Morristown, NJ |
| L-Leucine | Pharmachem, Kearny, NJ |
| Lupulone | Metagenics, Gig Harbor, WA |
| Lutein 10% VG | Kemin, Des Moines, IA |
| Oligonol | Maypro, Purchase, NY |
| Oxxynea | NB Consulting, Durham , NC |
| Panax Ginseng Ext | Naturex, South Hakensack, NJ |
| Puer Flower Extract | Toyo Bio-Pharma, Century City, CA |
| (Pueraria thomsonii) | |
| Resveratrol | InterHealth Nutraceuticals, |
| | Benicia, CA |
| Rhodiola rosea 3% Rosavins | Maypro, Purchase, NY |
| Rho-isoalpha acids (RIAA) | Metagenics, Gig Harbor, WA |
| Rosa Canina (Rosehips PMID | Amax NutraSource, Inc, Eugene, OR |
| 17400451) | |
| Rosemary Extract | Euromed, Pleasant Hill, CA |
| Rutin Powder | Seltzer, Carlsbad, CA |
| St. John's Wort 0.3% Gran | Euromed, Pleasant Hill, CA |
| Svetol (Coffee extract) | Berkem, New York, NY |
| Synephrine | Sigma, St. Louis, MO |
| Tetrahydroisoalpha acids | Metagenics, Gig Harbor, WA |
| (THIAA) | |
| Whey protein isolate | Glambia, Monroe, WI |
| (Provon 290) | |
| White Kidney Bean | AHD International, Atlanta, GA |
| Xanthohumol | Metagenics, Gig Harbor, WA |
| Xanthohumol Pure, | Metagenics, Gig Harbor, WA |
| Yerba mate PE 8% | P L Thomas, Morristown, NJ |
| Yohimbe extract 8% | Blue California, Rancho Santa |
| | Margarita, CA |

Results —Ranking 1-33 represents those test phytochemicals and extracts that significantly (p<0.05) activated AMPK relative to the DMSO solvent control (Table 2).

TABLE 2
Positive AMPK Activator Screening Results in C2C12 Myocytes

| | | | pAMPK/ |
|---|---|---|---|
| Rank | Test Material | Botanical Source | AMPK. |
| 1 | 6-Gingerol | Zingiber officinale | 3.52 |
| 2 | Fisetin | Cotinus coggygria | 2.77 |
| 3 | Synephrine | Citrus aurantium | 2.64 |
| 4 | Oligonol | grapes, apples, | 2.01 |
| | | persimmons | |
| 5 | Lupulone | Humulus lupulus | 1.63 |
| 6 | Whey protein isolate | Whey protein isolate | 1.61 |
| 7 | Chromium polynicotinate | — | 1.59 |
| 8 | Hexahydroisoalpha acids | Humulus lupulus | 1.57 |
| 9 | Xanthohumol | Humulus lupulus | 1.55 |
| 10 | Rho-isoalpha acids | Humulus lupulus | 1.52 |
| 11 | Elderberry Dry Ext. 4:1 | Sambucus | 1.51 |
| 12 | Gyrnnema sylvesre ext | Gymnema sylvesre | 1.46 |
| 13 | Black Tea Extract | Camellia sinensis | 1.45 |
| 14 | Insinase | Acacia nilotica | 1.45 |
| 15 | Applelin | Malus pumila | 1.44 |
| 16 | Black Currant PE 10% | Ribes nigrum L. | 1.44 |
| 17 | St. John's Wort 0.3% | Hypericum peiforatum | 1.44 |
| | Gran | | |
| 18 | Cocoanox 45% Polyphenols | Theobroma cacao | 1.42 |
| 19 | Blueberry Leaf PE | Vaccinium | 1.41 |
| 20 | Oxxynea | 10 fruits and | 1.39 |
| | | vegetables | |
| 21 | Black Tea PE 60% | Camellia sinensis | 1.34 |
| 22 | Rosa Canina (Rosehips) | Rosa canina | 1.34 |
| 23 | Isoalpha acids | Humulus lupulus | 1.33 |
| 24 | Cranberry | Vaccinium | 1.30 |
| | | erythrocarpum | |
| 25 | L-Leucine | — | 1.29 |
| 26 | Berberine | Hydrastis canadensis | 1.26 |
| 27 | Resveratrol | Vitis vinifera | 1.25 |
| 28 | Cascara Sagrada Bark | Rhamnus purshiana | 1.24 |
| | Powder | | |
| 29 | Epimedium PE 20% | Epimedium (horny Goat | 1.22 |
| | | Weed) | |
| 30 | BCM-95 | Curcuma longa | 1.21 |
| 31 | ID-alG | Opuntiaficus indica | 1.20 |
| 32 | Jambolean 10% | Syzygium cumini | 1.20 |
| 33 | Tetrahydroisoalpha | Humulus lupulus | 1.19 |
| | acids | | |

Unexpectedly, 26 of the test materials activated myocyte AMPK relative to the negative DMSO controls to a similar degree as the 1 mM AICAR positive control (Table 2) and three of these test materials were more active than AICAR (p<0.05). This unexpected result represents the first demonstration of phytochemicals or botanical extracts acting as AMPK-activating materials with greater potency than AICAR in any cell type.

### Example 3

### Select Gincer Extracts Activate AMPK in C2C12 Myocytes

Objective—The objective of this Example was to follow-up the results of Example 2 and assess the effect of three select ginger extracts on AMPK activation in C2C12 myocytes relative to the increase in pAMPK seen with 6-gingerol.

The Model—The C2C12 murine myocyte model as described in Example 1 was used in this example.

Chemicals, Cell Culture and Treatment—Chemicals, cell culture procedures, methods and statistical procedures used were as noted in Example 1.

Test Materials—Botanical extracts Ginger Extract (Suan Farma, Hackensack, N.J.), Ginger Root Powder (BI Nutraceuticals, Long Beach, Calif.), and Ginger Powdered Extract 6:1, Zingiber officinale (Draco, San Jose, Calif.) were used as the test materials and dosed at 25 μg/mL. The concentration for the positive control AICAR was 1.0 mM (338 μg/mL).

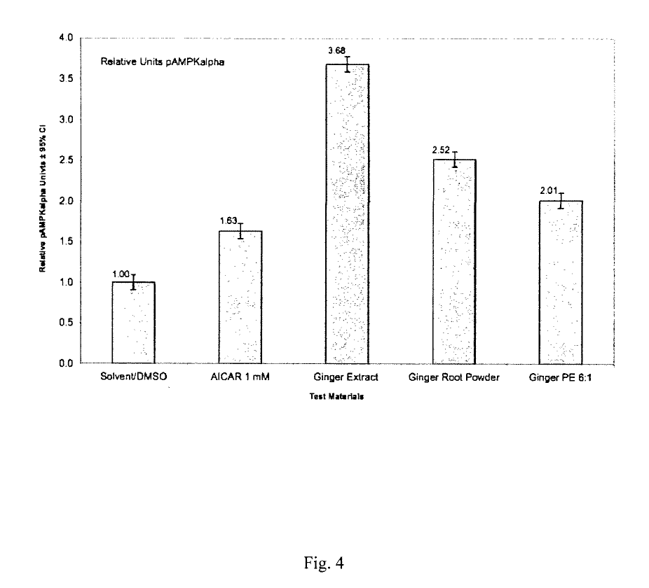

Results—All three test materials increased pAMPK relative to controls and more than the AICAR positive control (p<0.05, FIG. 4).

While the examples are limited to myocytes, it would be obvious to one of average skill in the art to modify these examples to apply to hepatocytes, adipocytes, cardiac cells or pancreatic cells.

The invention now having been fully described, it will be apparent to one of ordinary skill in the art that many further changes and modifications can be made thereto without departing from the spirit or scope of the appended claims.

1. A method for activating myocyte AMPK in an animal in need thereof, said method comprising administering to the subject in need a composition comprising a pharmaceutically effective dose of one or more of the members of the group consisting of phytochemicals or extracts isolated from Zingiber officinale, Cotinus coggygria, Citrus aurantium, Lupulone, Whey protein isolate, Chromium polynicotinate, Hexahydroisoalpha acids, Xanthohumol, Rho-isoalpha acids, Sambucu, Gymnema sylvesre, Camellia sinensis, Acacia nilotica, Malus pumila, Ribes nigrum L., Hypericum perforatum, Theobroma cacao, Vaccinium, Camellia sinensis, Rosa canina, Isoalpha acids, Vaccinium erythroCarpum, Leucine, Hydrastis Canadensis, Vitis vinifera, Rhamnus purshiana, Epimedium (horny Goat Weed), Curcuma longa, Opuntia ficus indica, Syzygium cumini, and Tetrahydroisoalpha acids.

2. The method of claim 1 wherein the activation of AMPK results in a decrease in mitochondrial membrane potential.

3. The method of claim 1 wherein the activation of AMPK results in an increase in resting energy expenditure.

4. The method of claim 1 wherein the activation of AMPK. results in a decrease in fat storage of visceral adipocytes.

5. The method of claim 1 wherein the activation of AMPK results in involves a decrease obesity and obesity complications.

6. The method of claim 1 wherein the activation of AMPK results in an increase in insulin sensitivity.

7. The method of claim 1 wherein the activation of AMPK results in a decrease in fasting hyperlipidemia.

8. The method of claim 1 wherein the activation of AMPK results in normalizing hypertension.
